(19) United States
(12) Patent Application Publication
(10) Pub. No.: US 2016/0041138 A1
(43) Pub. Date: Feb. 11, 2016

(54) Methods for Monitoring Airborne Contaminants and Agents using Atmospheric Condensate

(71) Applicants: Benny Pycke, Tempe, AZ (US); Isaac B. Roll, Tempe, AZ (US); Rolf Halden, Phoenix, AZ (US)

(72) Inventors: Benny Pycke, Tempe, AZ (US); Isaac B. Roll, Tempe, AZ (US); Rolf Halden, Phoenix, AZ (US)

(73) Assignee: Arizona Board of Regents on behalf of Arizona State University, Scottsdale, AZ (US)

(21) Appl. No.: 14/816,860

(22) Filed: Aug. 3, 2015

Related U.S. Application Data
(60) Provisional application No. 62/036,058, filed on Aug. 11, 2014.

(51) Int. Cl. G01N 33/00; G01N 1/22

(52) U.S. Cl. CPC G01N 33/0036; G01N 1/2273

(57) ABSTRACT

Methods for sample collection for assessment of air quality include generating an atmospheric condensate from air to be sampled, and collecting the atmospheric condensate in a collection vessel for subsequent use and analysis. Atmospheric condensate may be generated through a heat exchange module, such as a cold trap or the cooling coils of an HVAC system. The collection vessel may be an ultraclean glass jar or a cold trap. Air may be sampled actively or passively for both chemical and biological agents.

### CROSS-REFERENCE TO RELATED APPLICATIONS

This application claims priority from and is a non-provisional application of co-pending U.S. provisional application No. 62/036,058, filed Aug. 11, 2014, entitled “Methods and Systems for Monitoring Airborne Contaminants using Atmospheric Condensate.” U.S. application No. 62/036,058 is hereby incorporated by reference.

### TECHNICAL FIELD

The present invention relates to methods for monitoring airborne contaminants and signature compounds using atmospheric condensate, and, more particularly, the invention relates to a method for generating an atmospheric condensate from an HVAC system and collecting the atmospheric condensate in a collection vessel for determining the presence of contaminants and biological or chemical agents informing on behaviors, consumption, and human health hazards.

### BACKGROUND

Indoor air pollution has been identified as being among the top five environmental health risks (EPA Guide to Air Cleaners in the Home). Contaminants in the indoor air environment can occur as a mixture of gases and/or aerosols. These contaminants can originate from human activities inside or outside the building as well as from components of the building itself (e.g., off-gassing). Contaminants of concern include all types of hazardous substances: chemical, biological, and radiological agents. Environments of particular concern are those with little to no ventilation with outdoor air (including spacecraft, homes, and buildings); those with significant sources or little control over the sources of contamination (including submarines, aircraft, underground trains, mines, animal shelters, barns, storage units, commercial buildings, or homes close to industrial or contaminated sites); or those located in environments where ventilation may be warranted by weather conditions or undesirable levels of contaminants in indoor or outdoor air (including buildings in desert or polar environments or buildings close to industrial, waste, or contaminated sites).

Indoor air also represents a composite matrix signaling human activities (e.g., smoking), the consumption and handling of materials (e.g., nanomaterials), as well as the presence in indoor environments of chemical and biological threats (e.g., pathogenic bacteria, spores, fungi, viruses, infectious proteins and DNA or RNA. Monitoring indoor threats and signature compounds from a remote outdoor location can be of value in many applications, including occupational health, public health, and homeland security.

With Americans spending nearly 90% of their time indoors ^{[1]}, the quality of indoor air is of significant interest in understanding human exposure to airborne contaminants and the health effects thereof ^{[2,3]}. Indoor air is a dynamic matrix that carries a complex mixture of aerosols, larger suspended particles, and trace gases, all of which change over short- and long-term time scales in response to atmospheric conditions, human activities, material weathering, engineered environmental controls, regulatory changes, and other drivers ^{[4,5]}. Volatile organic chemicals (VOCs) of both indoor and outdoor origin have been consistently demonstrated to be present in higher concentrations indoors than outdoors ^{[6]}.

Indoor air quality is a composite measure of these numerous determinants, particularly the type and condition of building materials, indoor/outdoor air exchange rates, activities of indoor space occupants, and the operation of engineered systems used for environmental control, i.e., heating, ventilation, and air conditioning (HVAC) systems. The materials used to fabricate homes and consumer goods have changed significantly over recent generations, with the expected effect on the mixture of contaminants emitted into and detectable in indoor air ^{[7]}. Simultaneously, improvements in home energy efficiency have increased recirculation of indoor air and increasingly placed the burden of indoor-outdoor air exchange on the HVAC system ^{[8,9]}, particularly in the developed world.

HVAC systems (which include refrigeration and heat recovery ventilation systems) are common features in new construction in developed countries, with air conditioning systems being installed in more than 90% of new construction in some regions of the United States ^{[8]}. The principles of operation for both refrigeration and heat recovery ventilation include a heat exchanger that removes heat from warm and humid indoor air, condensing atmospheric moisture into a stream of liquid waste, which is routed out of the building. While the condensate stream largely consists of water recovered from atmospheric moisture, human respiration, and household activities, it is also a product of interactions with a mixture of trace gases and airborne particulate matter. Fractions of this complex mixture of chemicals are expected to condense on the cooled heat exchanger or partition from the atmosphere into the liquid accumulating thereon, with the fluid stream acting as a trap for airborne contaminants. Hence, the collection of indoor air condensate allows for space and time integrated sampling of the indoor air, since the air handler of the ventilation system supplies large volumes of air from multiple rooms to the heat exchanger.

At present, indoor air chemistry is typically characterized by analysis of whole air samples, cryogenic air traps and sorptive samplers ^{[5,10-12]}. These approaches enable detection of VOCs, semivolatile organic compounds (SVOCs), and less-volatile organic chemicals typically associated with particulate matter down to parts-per-trillion (ppt) concentrations. Such samples can be obtained either discretely in time, or over extended durations to facilitate time-integrated air quality assessments. These methods provide information that is spatially discrete within a building, and require access to the indoor environments under investigation. Analysis of dust and particulate matter ^{[13-17]} provides another avenue for investigating human exposure to inhalable environmental contaminants, particularly the less volatile species. These standard methods typically require access to the sampled building and are collected in discrete locations inside the building; hence, there is an opportunity to investigate the applicability of new methods that are non-intrusive and allow for time and space integrated sampling.

In contrast to conventional methods, the inventors here present a new, economical and promising approach to characterizing indoor air contaminant mixtures by sampling condensate from HVAC systems. Unlike known techniques, the novel method disclosed here is spatially integrated over a whole accessible living space, and does not require access to the interior of a home. As further shown herein, the disclosed method was demonstrated to detect a wide range of volatile, semivolatile, and low-volatility anthropogenic substances in indoor air. In this work, the mixture of contaminants detected was not influenced by the filterable fraction of particulate matter in air; rather, it was the product of gas phase and submicron (i.e., inhalable) contaminants that are readily available through the inhalation route to building occupants. Detected mixtures were largely consistent over short time spans for individual buildings, identifiably different between buildings, and sensitive to the introduction of consumer products to the living space. One significant difference between the data provided by conventional air sampling techniques and the condensate sampling technique presented here is the ability to predict concentrations in the bulk indoor air.

Further in differentiation from conventional techniques, for the first time the present disclosure provides a method that enables the indirect, qualitative monitoring of air quality from buildings and living spaces at scales both large (via catchments for condensate from entire floors or buildings) and small (condensate from single family homes or apartments) without requiring access to the interior of the building. Detailed herein is the analysis of HVAC condensate samples by liquid and gas chromatography mass spectrometry and tandem mass spectrometry demonstrating the feasibility of detecting indoor air contaminants across a generous spectrum of hydrophobicities and volatilities.

### BRIEF SUMMARY OF THE DISCLOSURE

This summary is provided to introduce, in a simplified form, a selection of concepts that are further described below in the Detailed Description. This summary is not intended to identify key features of the claimed subject matter, nor is it intended to be used as an aid in determining the scope of the claimed subject matter.

Provided herein is a method of sample collection for assessment of air quality in a structure including circulating indoor air through HVAC ductwork; filtering the indoor air to remove a portion of air contaminants; generating an atmospheric condensate from the filtered air; collecting the atmospheric condensate in a collection vessel; and conducting an analysis of the atmospheric condensate. Air may be sampled actively or passively for both chemical and biological agents.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1 representatively illustrates sampling of atmospheric condensate using the HVAC system of a building or other environment with a HVAC system.

FIG. 2 representatively illustrates sampling of atmospheric contaminants using an air sampler unit.

FIG. 3 representatively illustrates approximate locations of condensate sample collection sites in Maricopa County, Arizona.

FIG. 4 graphically illustrates Gas chromatograms representing samples derived from House A, House B, and a reagent blank.

FIG. 5A-FIG. 5C graphically illustrate gas chromatograms for condensate samples taken at different dates and under different conditions from a selected site.

FIG. 6 graphically illustrates a Range of volatilities (expressed as Boiling Point, BP) and hydrophobicities (as Octanol-Water Partitioning Coefficient, log Kow) of compounds identified in HVAC condensate.

FIG. 7 graphically illustrates histograms illustrating the number of compounds detected within a given range of (A) boiling points and (B) log Kow values. Boiling Point (BP) and Octanol-Water Partitioning Coefficient (log Kow) predicted by ACD/Labs.

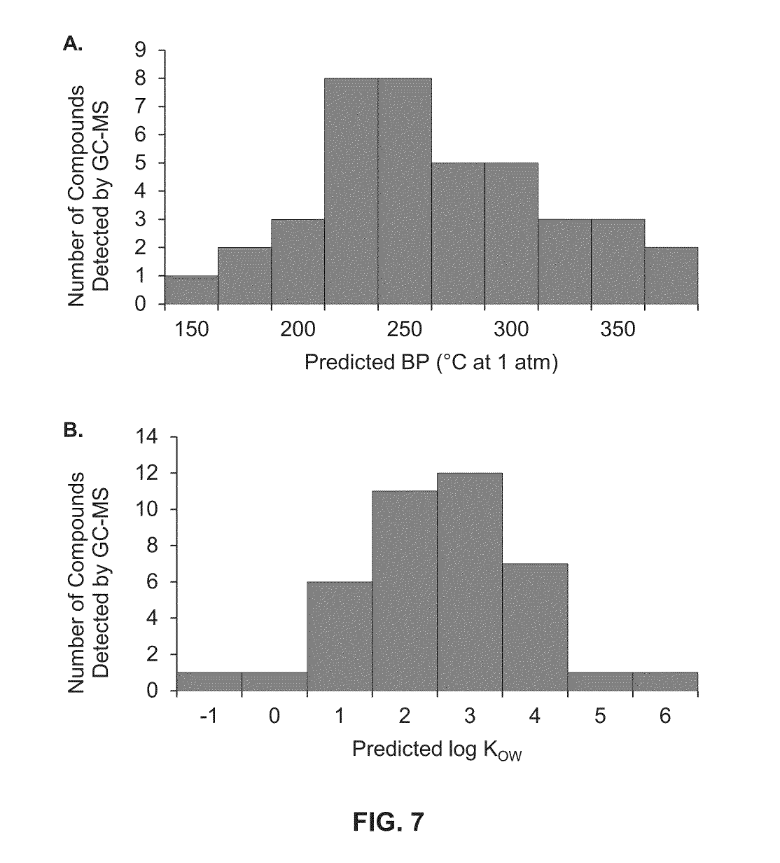

FIG. 8 shows Table 2 which tabulates anthropogenic compounds detected and confirmed by GC-MS in HVAC condensate, sorted by molecular weight.

In the drawings, identical reference numbers identify similar elements or components. Elements and steps in the figures are illustrated for simplicity and clarity and have not necessarily been rendered according to any particular sequence. For example, steps that may be performed concurrently or in different order are illustrated in the figures to help to improve understanding of embodiments of the present invention. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION OF EXEMPLARY EMBODIMENTS

The following disclosure describes a method of sample collection for assessment of air quality. Several features of methods and systems in accordance with example embodiments are set forth and described in the figures. It will be appreciated that methods and systems in accordance with other example embodiments can include additional procedures or features different than those shown in the figures. Example embodiments are described herein with respect to a method for generating an atmospheric condensate from an HVAC system and collecting the atmospheric condensate in a collection vessel for the presence of contaminants. However, it will be understood that these examples are for the purpose of illustrating the principles, and that the invention is not so limited.

### DEFINITIONS

Generally, as used herein, the following terms have the following meanings when used within the context of microarray technology:

The articles “a” or “an” and the phrase “at least one” as used herein refers to one or more. Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or combinations and/or variations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “plurality” is understood to mean more than one. For example, a plurality refers to at least two, three, four, five, ten, 25, 50, 75, 100, 1,000, 10,000 or more.

As used in this specification, the terms “processor” and “computer processor” encompass a personal computer, a tablet computer, a smart phone, a microcontroller, a microprocessor, a field programmable object array (FPOA), a digital signal processor (DSP), an application-specific integrated circuit (ASIC), a field programmable gate array (FPGA), a programmable logic array (PLA), or any other digital processing engine, device or equivalent capable of executing software code including related memory devices, transmission devices, pointing devices, input/output devices, displays and equivalents.

As used herein, “HVAC” means engineered systems used for environmental control in a building or other structure, i.e., heating, ventilation, and air conditioning systems.

As used herein, “indoor air”, encompasses air contained within a living quarters or other contained environment such as a building, airplane, space craft, ship, submarine or the like.

“Obtaining” is understood herein as manufacturing, purchasing, or otherwise coming into possession of.

As used herein, “agent”, encompasses any chemical compound or biological material, including microorganisms, informing on the presence or occurrence of any hazard, behavior, consumption or other activity, which is taking place in the indoor environment or recently has been taking place there.

### EXAMPLE EMBODIMENTS

The methods for monitoring airborne contaminants using atmospheric condensate comprise generating, collecting, and analyzing atmospheric condensate to provide a quantitative or qualitative assessment of air quality. The methods allow for easy, economical, and rapid sampling of air quality in closed, semi-closed, or open-air environments where air quality may be of concern for environmental health, environmental quality, law enforcement, or homeland security. Thus the methods enable both indoor and outdoor locations to be sampling sites. The technician responsible for the sampling or monitoring does not need to be on location or enter the premises when using the existing HVAC heat exchange and/or duct work, so the methods allow for sentinel, remote, non-intrusive sampling.

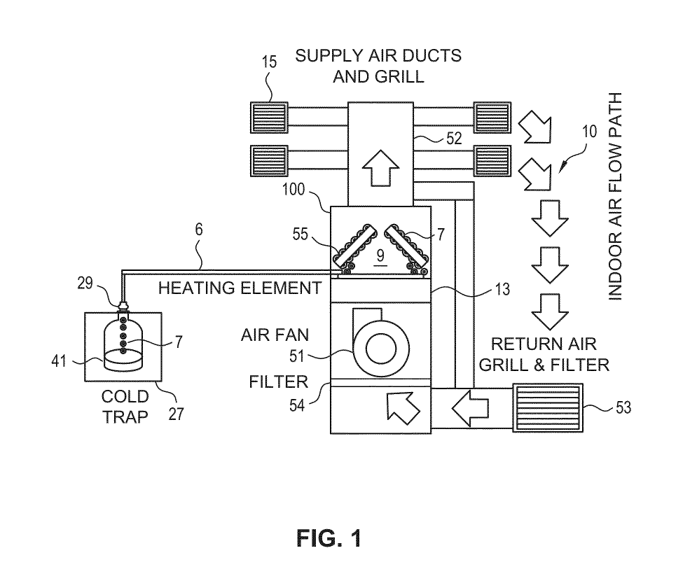

Referring now to FIG. 1, sampling of atmospheric condensate using the HVAC system of a building or other environment with a HVAC system is representatively illustrated. It will be understood that sampled environments can include airplanes, boats, submarines, and spacecraft, for example. An HVAC system 100 includes an air fan 51 that recirculates indoor air through a heating element 13 and the HVAC ductwork 52, as indicated by arrows 10. This causes the indoor air to pass through one or more air filters 53, 54 that remove some of the indoor air contaminants. These filters can be retrieved for analysis of aerosols and aerosol-borne contaminants. Following filtration, the air comes into contact with the cooling coils 55 of the HVAC system and atmospheric condensate 7 is created on the surfaces of the cooling coils 55. The accumulated condensate 7 trickles down the coils 55 into a collection pan 9 and is discharged via the condensate evacuation spout 6, where it is passed through a filter, as, for example, an SPE cartridge 29. The collected condensate can be filtered prior to passing into the recipient or through the SPE cartridge 29. After the collected condensate 7 is filtered it is collected for analysis. At the collection site, the sample can be collected in a cooled glass recipient, or cold trap 27 or it can be extracted on location using a solid-phase extraction (SPE) cartridge instead of the cold trap (not shown).

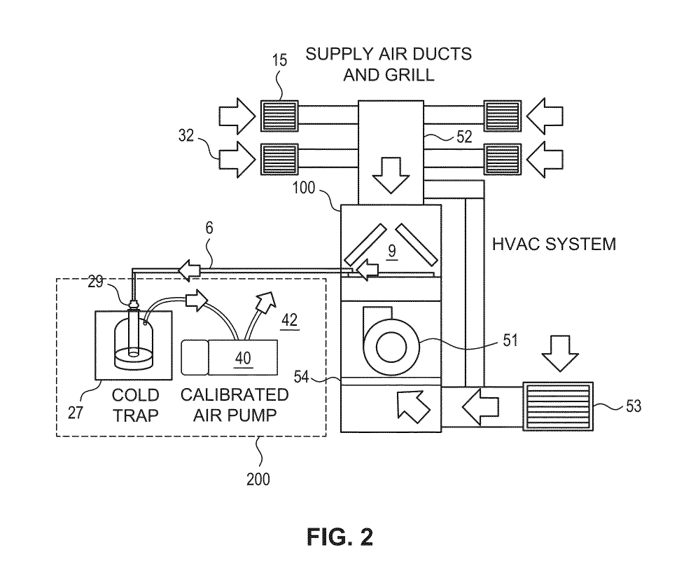

Referring now to FIG. 2, sampling of atmospheric contaminants using an air sampler unit including a cold trap and a calibrated air pump is schematically illustrated. An air sampler unit 200 can either be connected to the HVAC ducts 52 of a building or other environment with an HVAC system 100 via a condensate spout 30 (as shown here) or deployed independently inside or outside of a building (not shown). It will be understood that sampled environments can include airplanes, boats, submarines, and spacecraft, for example. The action of the air pump will pull air in as indicated by arrows 32 from across the building via the HVAC ducts 52 through the condensate spout, through a filter 29, via the cold trap 27 through a pump 40, and out into the environment as indicated by arrow 42. In one example the air pump 40 may advantageously be a calibrated air pump. A preferential flow will be created from HVAC grills 15 that do not have a filter because of the resistance encountered by the filters, so it is recommended to remove air filters prior to sampling. The cold trap 27 can be filled with a solvent 41 or kept empty when starting the sampling and the tube in the cold trap can be positioned into the solvent to improve capturing efficiency. The pump exhaust (indicated by arrow 42) is vented outside of the sampled environment (e.g., building) to prevent recirculation of contaminant-purged air. The inlet into the cold trap can be outfitted with a glass fiber filter to capture particulate contaminants. This filter can be analyzed separately from the solvent or produced atmospheric condensate.

### EXAMPLES

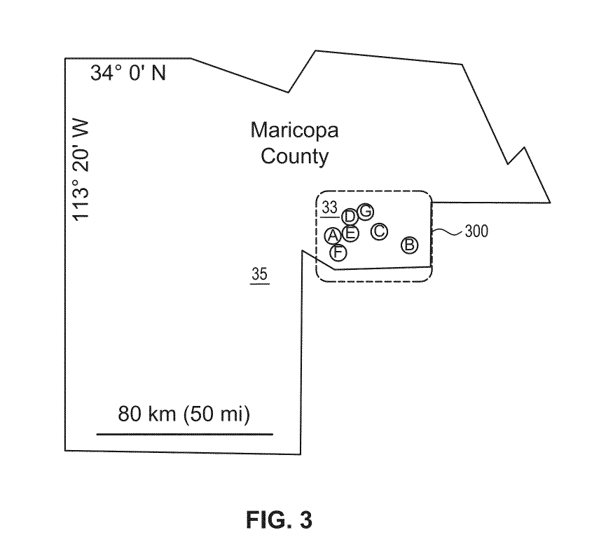

Referring now to FIG. 3, approximate locations of condensate sample collection sites in Maricopa County, Arizona are representatively illustrated. Samples were collected from six residences and one business A-G in Maricopa County, Arizona during August and September of 2013. All seven buildings have central air conditioning with condensate drains installed in easily accessed locations. These buildings were constructed between 1960 and 2012, and were distributed over an area of more than 1000 km2 as indicated by the broken line region 300. Participants were provided instructions for sampling and sample handling. Participants were also provided with new, unused dust filters to install in their air handlers, to ensure proper air flow through the heat exchanger, and to reduce any effect of accumulated particulate contamination on the condensate contaminant mix. However, the installation of clean filters was not mandatory or recorded.

Condensate samples were collected to provide information about the unique chemistry of individual buildings in the seven locations A-G, changes in the mixture of indoor air contaminants detected over the course of several days, and the effects of occupancies and human activities in the home on the chemical composition of collected condensate. Typically, there were three sampling dates per location. Sampling was carried on the basis of one location per parameter.

### Sampling Procedure

Decontaminated 1.0-gallon (3.8-liter) glass bottles were placed beneath outdoor HVAC condensate drain spouts such that the condensate dripped directly into the bottle with no physical contact between the spout and the bottle. The volume of condensate recovered in the sampling period varied from 600 to 3800 mL. The production of HVAC condensate was noted to be greater on days with higher atmospheric humidity.

The bottle, and the interface between the opening of the bottle and the spout were covered with decontaminated (that is muffle furnace-baked) aluminum foil to protect the sample from sunlight, mitigate sample evaporation, prevent sample contamination due to atmospheric deposition of particulates, and to exclude natural outdoor condensation (e.g., rain and dew) from the sample. If rain was documented during the sampling period, the sample was discarded.

The samples from homes were typically collected overnight to maximize the number of inhabitants and minimize indoor/outdoor air exchanges through doors and windows. The sample from a business was collected during peak operating hours. Upon cessation of sampling, the bottle was capped, immediately refrigerated with ice packs and shipped to the laboratory for storage at 4° C., and subsequent processing (i.e., aliquoting, weighing, filtering, and extraction) within 24 h. consisting of 1.0-liter glass bottles of ultrapure water were exposed to the indoor and outdoor atmosphere for 12 h via brass swan-neck tubes, permitting interaction of the bottle headspace with the atmosphere while mitigating the intrusion of suspended aerosols and particulates. At each home, one field blank was located adjacent to the exterior HVAC condensate spout and another placed near the air handler intake within the building. Trip blanks of identical volume accompanied the field blanks but were not exposed to the atmosphere at the sampling sites.

Within one day of sampling, all samples were homogenized by rotary shaking and split in two equal-volume subsamples based on gravimetric analysis. One volume was extracted as collected (“unfiltered”), and the other (“filtered”) represented processed condensate, filtered using a decontaminated vacuum filtration assembly (Sigma-Aldrich) with 47 mm GF/F 0.6-0.8 m, borosilicate glass fiber filters (Whatman). Filters were replaced at intervals of approximately 250 mL of concentrate to ensure rapid flow of all samples and to minimize losses of volatile contaminants during vacuum filtration. After filtration, both filtered and unfiltered condensate subsamples were transferred to decontaminated glass receptacles and immediately extracted.

### Solid-Phase Extraction

All samples were extracted using an automated offline solid-phase extraction (SPE) apparatus (Dionex Autotrace 280, Thermo Fisher Scientific Inc., Sunnyvale, Calif.) using Strata C18-E SPE cartridges (Phenomenex, Torrance, Calif.) with 500 mg sorbent. Aliquots of up to 1000 mL of condensate were automatically loaded onto the cartridge, which was then eluted with 5 mL of MS-grade methanol.

### Analysis by LC-MS/MS

Sixteen samples from seven buildings were analyzed on a liquid chromatograph (LC; Prominence, Shimadzu Corp., Kyoto, Japan) with autosampler coupled to a tandem mass spectrometer (MS/MS) with electrospray ionization (API 4000, AB SCIEX, Framingham, Mass.). The analytes of interest were separated on a 4.6×150-mm C8 column (X-Bridge, Waters, Milford, Mass.) with 3.5-m particle size preceded by an equivalent guard column. The MS/MS was programmed to perform multiple reaction monitoring of a suite of ten anthropogenic compounds relevant to human biomonitoring studies ^{[19-23]}, a manufacturing impurity and two human metabolites associated with one of the compounds, four carbon-13 labeled surrogates, and a laboratory control compound. The control compound, 4-methylumbelliferone (MUF), had been used extensively in this laboratory as a component of other LC-MS/MS studies, and would provide an indication of cross-contamination from preparatory surfaces and instruments.

### Analysis by GC-MS

The same sixteen samples, and an additional two that were prepared subsequent to LC-MS/MS analysis, were analyzed on a gas chromatograph (GC; Model 7890, Agilent Technologies, Santa Clara, Calif.) equipped with an autosampler (MPS, GERSTEL GmbH & Co., KG, Mülheim an der Ruhr, Germany) coupled to a tandem mass spectrometer (MS/MS; Model 7000, Agilent Technologies, Santa Clara, Calif.). A DB-5MS column (30-m long×0.250-mm inner diameter×25-m film thickness; Agilent Technologies, Santa Clara, Calif.) was used with helium carrier gas. The MS was operated in scan mode over an m/z range of 50-300 with a dwell time of 500 ms. The National Institutes of Standards and Technology (NIST) Mass Spectral Search Program (Version 2.0f) with the NIST/EPA/NIH Mass Spectral Library (NIST 08) was used for compound identification, and descriptors of the most likely candidate compound (including percent match, CAS number, and retention time) were recorded as a database entry. The instrument parameters for the GC-MS method are provided in Table S5 of the Supplementary material.

Potential Analysis of Biological Agents

In addition to the assays described above, other additional assays to be conducted on indoor condensate may include the determination of biological agents and constituents of the same, taken from the group consisting of bacteria, fungi, yeasts, molds, viruses, parasites, prions, DNA, and RNA, using methods understood and available to those skilled in the art of chemical and biological monitoring (e.g., [28-29]).

### Quality Control

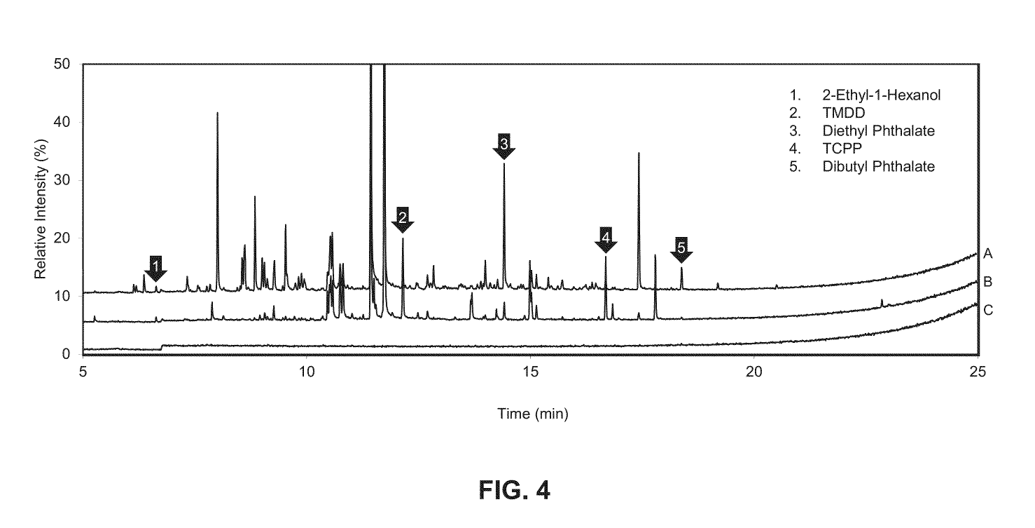

Referring now to FIG. 4, gas chromatograms representing samples derived from House A, House B, and a reagent blank are graphically illustrated. Traces A, B from the two houses (of seven structures studied) illustrate the shared and the unique chemistry of the HVAC condensate produced. Trace C represents the reagent bank readings. Five contaminant compounds which have been confirmed with authentic standards are indicated with black arrows 1-5. The relative intensity has been scaled to half of the most abundant peak to show finer detail, and the chromatograms for Houses A and B have been offset by +10 and +5%, respectively, for clarity.

The standards are:

1. 2-Ethyl-1-Hexanol,

2. TMDD,

3. Diethyl Phthalate,

4. TCPP, and

5. Dibutyl Phthalate.

Trip blanks and indoor and outdoor field blanks were provided for two of the locations sampled during the study, and showed no contamination during LC-MS/MS or GC-MS analysis. Extractions of unadulterated ultrapure water were performed with each batch of samples. None of these reagent blank samples (i.e., procedural blanks) showed contamination during LC-MS/MS or GC-MS analysis, and the signal/noise ratios never exceeded 3 for the 16 specific mass transitions included in the LC-MS/MS program. The laboratory contamination control compound MUF was not detected in any samples analyzed by the LC-MS/MS, indicating that contamination from laboratory instruments or surfaces was unlikely.

### Targeted Survey of Endocrine Disrupting Compounds

Analysis by LC-MS/MS demonstrated the presence of all ten targeted endocrine disrupting compounds (EDCs) in at least one of the 16 HVAC condensate samples (as shown in Table 1). The insecticide fipronil and antimicrobial triclosan (TCS) were detected in all samples (100%). Five parabens (methyl-, ethyl-, propyl-, butyl-, and benzyl-) commonly used as preservatives in personal care and food products were detected in 14-16 samples (88-100%), with propylparaben and butylparaben being ubiquitous (100%). Triclocarban (TCC), which is a compound commonly used in antimicrobial soaps, and its tetrachlorinated manufacturing impurity 3′-Cl-TCC were detected (and co-occurred) in half (8 of 16, 50%) of the samples analyzed. The brominated flame retardant tetrabromobisphenol-A (TBBPA) was detected in nearly half (7 of 16, or 44%) of the samples, whereas its non-brominated congener, bisphenol-A (BPA), which is a common component of polycarbonate plastics, was detected in only two samples (13%).

Two additional human metabolites of TCC, 2′-hydroxytriclocarban (2′-OH-TOO) and 3′-hydroxytriclocarban (3′-OH-TCC) were screened for but never detected in condensate extracts (IDLs of 9 and 10 ng/L, respectively). Lack of detection of these human metabolites of the antimicrobials was expected, as these compounds are excreted in the urine and stool, whereas the TCC detected in the condensate samples would be expected to be a product of the storage and topical application of TCC-containing products.

TABLE 1
Suite of compounds targeted for detection by LC-MS/MS in submicron-
filtered HVAC condensate, sorted by molecular weight.

| | | | BP | log | No. detected |
|---|---|---|---|---|---|
| Compound | Source | MW^{a} | (° C.)^{b} | K_{OW}^{c} | (Percentage) |
| Paraben, methyl- | Preservative | 152 | 266 | 1.9 | 14 (88) |
| Paraben, ethyl- | Preservative | 166 | 297 | 2.4 | 15 (94) |
| Paraben, propyl- | Preservative | 193 | 329 | 2.8 | 16 (100) |
| Paraben, butyl- | Preservative | 194 | 309 | 3.5 | 16 (100) |
| BPA | Plastic | 228 | 401 | 3.4 | 2 (13) |
| | monomer | | | | |
| Paraben, benzyl- | Preservative | 228 | 390 | 3.6 | 15 (94) |
| TCS | Antimicrobial | 290 | 345 | 5.2 | 16 (100) |
| TCC | Antimicrobial | 316 | 344 | 5.7 | 13 (81) |
| Fipronil | Insecticide | 437 | 510 | 4.8 | 16 (100) |
| TBBPA | Flame retardant | 544 | 418 | 7.3 | 7 (44) |

BPA is bisphenol A,
TCS is triclosan,
TCC is triclocarban, and
TBBPA is tetrabromo-bisphenol A.
^{a}Molecular weight.
^{b}Boiling point at 1.0 atm predicted by ACD/Labs.
^{c}Octanol-water partitioning coefficient predicted by ACD/Labs.

### Non-Targeted Survey of Organic Contaminants

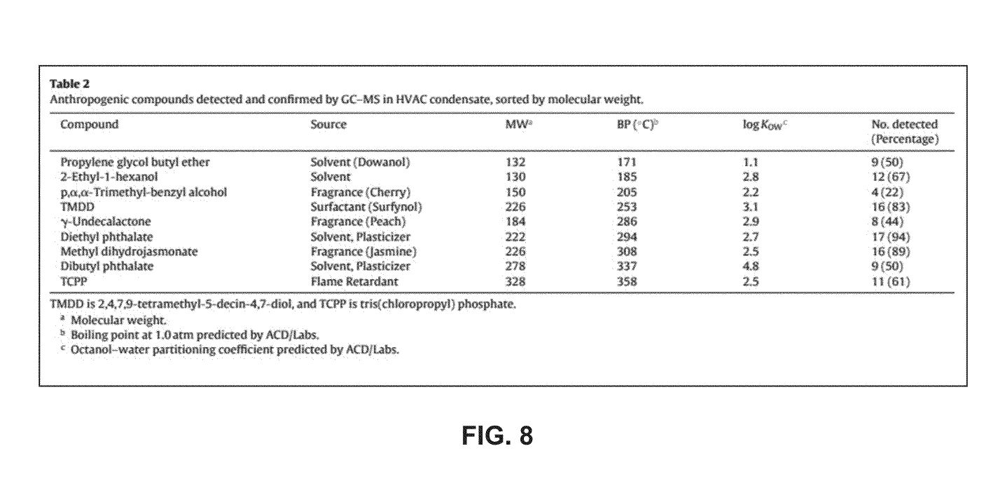

Eighteen samples were analyzed by GC-MS in full scan mode (m/z 50-300). Seventeen samples (16 previously analyzed by LC-MS/MS and one subsequently prepared) were used for a non-targeted survey of organic contaminants; the eighteenth was prepared specifically to investigate the effect of the introduction of a new household chemical (as described below), and was excluded from the non-targeted survey. The survey samples yielded an average of 33 chromatographic peaks (n=17) at a total of 112 unique retention times. Approximately 85% of these peaks were tentatively identified by their corresponding mass spectra. A subset of samples was analyzed in scan mode over an m/z range of 50-600, but as it did not yield additional peaks, the more sensitive m/z 50-300 data was used for all subsequent analysis. A robust subset of the data was selected by retaining components that; (i) were detected at unique retention times, (ii) occurred in multiple samples, and (iii) corresponded to compounds for which standards were commercially available. A total of 40 unique, tentatively identified compounds remained. To evaluate the quality of the tentative identifications, we performed a randomized analysis of 25% of these signal-producing entities by comparing their retention times and mass spectra to those of commercial, authentic standards. Among the 10 randomly selected compounds detected in condensate, the identity of nine (90%) was confirmed unambiguously using this approach (as shown in FIG. 8, Table 2). Positively identified compounds included common fragrances, solvents, and the chlorinated organophosphate flame retardant TCPP.

### Contaminant Mixtures Detectable in Condensate as a Function of Sampling Location and Human Activity

A comparison of chromatograms of contaminant mixtures present in condensate from different homes readily revealed some marked differences as well as similarities between buildings (See FIG. 4). These may be partly ascribed to the manner of operation of the HVAC system (e.g., duration of use) and the physical layout of the building. The single sample taken from a commercial structure, for example, exhibited the fewest detectable contaminants (11 peaks recorded) when analyzed by GC-MS. This is suspected to be an effect of dilution of the liquid stream due to higher condensate production, resulting from the larger capacity of the system, larger enclosed space, and different ventilation requirements of a commercial system.

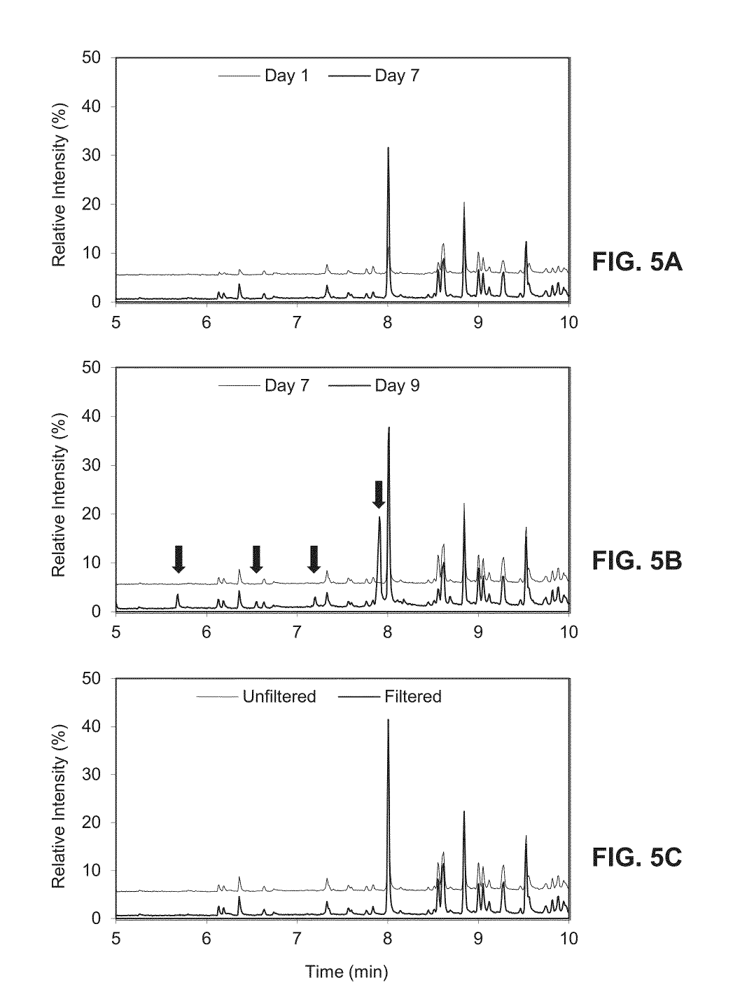

Referring now to FIG. 5A-FIG. 5C, there graphically illustrated are (A) gas chromatograms for condensate samples taken at different dates from House A showed consistency in the chemical mixtures present and revealed differences in the strength of detected signals, (B) controlled introduction of a new household product (in this case, burning of a scented candle) generated new, easily discerned peaks (indicated by vertical arrows) when comparing chromatograms taken from condensate samples before and after the product was used, and (C) example GC-MS chromatograms resulting from the analysis of unfiltered and filtered condensate from one of the study locations. The relative intensities have been scaled to half of the most abundant peak (not shown) to show finer detail and one chromatogram offset +5% for clarity.

As can be seen from the chromatograms, the present study demonstrates that condensate chemistry is directly impacted by human activities in the buildings. Repeated sampling of the same buildings on different dates demonstrated that the mixture of contaminants detected in each building (i.e., the indoor contaminant fingerprint) was largely consistent over time (as shown in FIG. 5A). Yet the introduction of new household activities was immediately apparent in GC-MS chromatograms when the occupants of one home were asked to burn a scented candle at least 20 feet away from the HVAC intake vent (as shown in FIG. 5B). These participants were known to not have used such products, and the additional activity produced new, easily discernable peaks when comparing chromatograms from before and after the requested activity.

### Results not Affected by Submicron Filtration

One of the critical questions when assessing the relevance of HVAC condensate for monitoring indoor air quality and the associated human exposures is the transport mechanism by which contaminants enter the liquid stream. To address this question, extracts were prepared with and without submicron filtration of the condensate sample prior to SPE. For unfiltered samples, contaminant mass associated with particulate matter would be physically retained on the SPE cartridge, and expected to be eluted by the organic solvents used during solid phase extraction, leading to the appearance or magnification of peaks for contaminants that did not previously partition effectively from the particulate matter into the condensate. The flame retardant TCPP, detected here in condensate and previously in both particle and vapor phases ^{[24]}, would be expected to demonstrate this effect if it occurs.

However, the comparison of chromatograms of all filtered and unfiltered samples consistently failed to demonstrate such an artifact (as shown in FIG. 5C), suggesting that the contaminant contribution from particles greater than approximately 0.7 m was negligible for the contaminants detected in this study.

While TCC, TCS, and most of the parabens have boiling points that suggest low volatility, they nevertheless were detectable in the condensate samples. All of these compounds are common components of personal care products. Their detection in condensate may be facilitated by volatilization or aerosolization (e.g., during showering). Typically, TCS, BPA, and TBBPA exist in household dust at ng- and μg-per-g concentrations ^{[15]}. Yet the ubiquitous detection of TCS in this study is noteworthy given its low volatility and the fact that; (i) sampling occurred downstream of the air filtration unit of the HVAC systems, (ii) many samples were collected after installation of clean air filters, (iii) none of the participants reported showering during the sampling period, and (iv) the condensate samples were filtered prior to extraction.

Furthermore, the ubiquitous detection of TCS occurred without regard to the age of construction of the monitored buildings, which were constructed over a five decades between 1960 and 2012. This is noteworthy as the age of construction impacts both the type of building material used (including the introduction of antimicrobial building materials ^{[25]}) and the accumulation of dust and contaminants in the air handling system. Thus, multiple lines of evidence point to gas phase and fine (submicron) aerosols as the source of the analytes detected; yet, future studies should substantiate the extent to which these chemicals occur as volatiles or associated with submicron aerosols to assess the relevance for human exposures of this plausibly inhalable fraction.

### Hydrophobicity and Volatility Range of Analytes

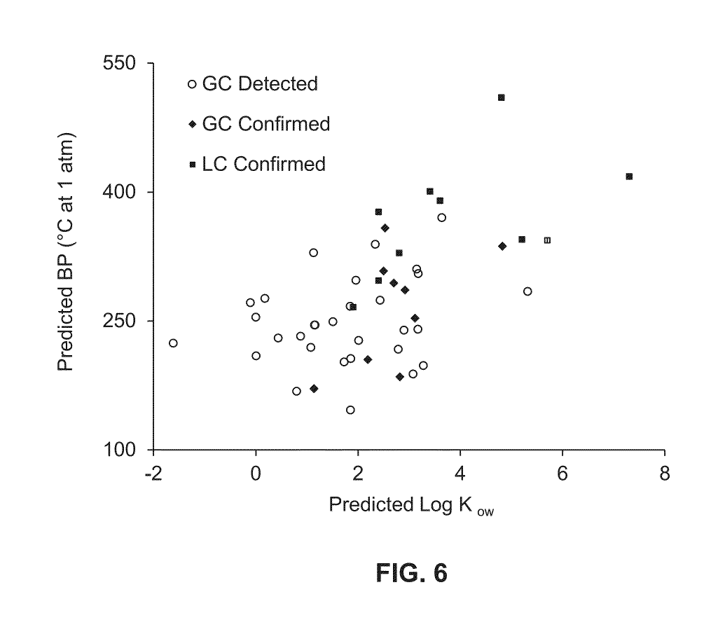

Referring now to FIG. 6, a range of volatilities (expressed as Boiling Point, BP) and hydrophobicities (as Octanol-Water Partitioning Coefficient, log KOW) of compounds identified in HVAC condensate is graphically illustrated. The thermodynamic properties of indoor air contaminants would be expected to drive the mixture found in condensate, with boiling point and octanol-water partitioning coefficients being chief among them. To better understand the effect of these physical properties, predicted values were tabulated (See, for example, Table 1) and plotted for all of the compounds targeted by LC-MS/MS and those tentatively or positively identified by GC-MS/MS. For data consistency, chemical-physical data for chemicals were taken from the ACD/Labs suite of chemical property predictive software. For the compounds reported in this study, predicted boiling points ranged from less than 150° C. to more than 500° C. suggesting that the spectrum of air pollutants suitable for monitoring in condensate may extend far beyond the limited range of volatile compounds featuring boiling points at or below ambient air temperature. Similarly, the compounds investigated in this study covered a significant spectrum of hydrophobicity, indicated by predicted log KOW values ranging from −2 to nearly 8.

Referring now to FIG. 8, there Table 2 tabulates anthropogenic compounds detected and confirmed by GC-MS in HVAC condensate, sorted by molecular weight. Non-targeted condensate surveys, such as the one presented here, will be biased toward an optimal hydrophobicity and boiling point associated with the specific sample collection, extraction, and analysis methods. For the GC-MS data presented (n=40), a central tendency for predicted boiling point is demonstrated around a mean of 252° C. with a standard deviation of 54° C., and similarly for predicted log KOW around a mean of 2.0 with a standard deviation of 1.4. Available data suggest that more hydrophobic or acidic contaminants were not a substantial component of the collected condensate samples, since non-targeted GC-MS analysis of subsequent toluene elution of the C18 cartridges and acidification of the condensate sample prior to SPE, respectively, did not yield additional chromatographic peaks in the GC-MS (results not shown). The spectrum of indoor air pollutants to be monitored using condensate may potentially be expanded, however, by modifying the protocol to include refrigeration of sampling containers, the use of different stationary extraction phases, elution with different solvents, and analysis by employing alternative or additional methods such as liquid chromatography-high resolution mass spectrometry (LC-HRMS).

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by different equipment, and devices, and that various modifications, both as to the equipment details and operating procedures, may be accomplished without departing from the true spirit and scope of the present invention. Accordingly, the scope of the invention should be determined by the claims and their legal equivalents rather than by merely the examples described. For example, potential exists for the methods disclosed herein to be used in the screening of living spaces for contaminants of concern, for surveys of large numbers of living spaces, and for monitoring the changes in indoor air quality associated with aging of construction materials.

Note that the steps recited in any method or process claims may be executed in any order and are not limited to the specific order presented in the claims. Additionally, the components and/or elements recited in any apparatus claims may be assembled or otherwise operationally configured in a variety of permutations and are accordingly not limited to the specific configuration recited in the claims.

### REFERENCES

The teachings of the following publications are incorporated herein in their entirety by this reference.
[1] N. E. Klepeis, W. C. Nelson, W. R. J. P. Ott Robinson, A. M. Tsang, P. Switzer, J. V. Behar, S. C. Hern, W. H. Engelmann, The national human activity pattern survey (NHAPS): a resource for assessing exposure to environmental pollutants, J. Expos. Sci. Environ. Epidemiol. 11 (2001).
[2] P. N. Breysse, R. J. Delfino, F. Dominici, A. C. P. Elder, M. W. Frampton, J. R. Froines, A. S. Geyh, J. J. Godleski, D. R. Gold, P. K. Hopke, P. Koutrakis, N. Li, G. Oberdorster, K. E. Pinkerton, J. M. Samet, M. J. Utell, A. S. Wexler, US EPA particulate matter research centers: summary of research results for 2005-2011, Air Qual. Atmos. Health 6 (2013) 333-355.
[3] C. Billionnet, E. S. Gay Kirchner, B. Leynaert, I. Annesi-Maesano, Quantitative assessments of indoor air pollution and respiratory health in a population-based sample of French dwellings, Environ. Res. 111 (2011) 425-434.
[4] L. E. LaRosa, T. J. Buckley, L. A. Wallace, Real-time indoor and outdoor measurements of black carbon in an occupied house: an examination of sources, J. Air Waste Manage. Assoc. 52 (2002) 41-49.
[5] K. Dettmer, W. Engewald, Ambient air analysis of volatile organic compounds using adsorptive enrichment, Chromatographia 57 (2003) S339-S347.
[6] S. Fuselli, M. De Felice, R. Morlino, L. Turrio-Baldassarri, A three year study on 14 VOCs at one site in Rome: levels, seasonal variations, indoor/outdoor ratio and temporal trends, Int. J. Environ. Res. Publ. Health 7 (2010) 3792-3803.
[7] O. A. Seppaänen, W. J. Fisk, Summary of human responses to ventilation, Indoor Air 14 (2004) 102-118.
[8] C. J. Weschler, Changes in indoor pollutants since the 1950s, Atmos. Environ. 43 (2009) 153-169.
[9] R. M. Lazzarin, A. Gasparella, Technical and economical analysis of heat recovery in building ventilation systems, Appl. Therm. Eng. 18 (1998) 47-67.
[10] D. K. W. Wang, C. C. Austin, Determination of complex mixtures of volatile organic compounds in ambient air: canister methodology, Anal. Bioanal. Chem. 386 (2006) 1099-1120.
[11] R. K. M. Jayanty, Evaluation of sampling and analytical methods for monitoring toxic organics in air, Atmos. Environ. 23 (1989) 777-782 (1967).
[12] M. R. F. Ras Borrull, R. M. Mame, Sampling and preconcentration techniques for determination of volatile organic compounds in air samples, Trends Anal. Chem. 28 (2009) 347-361.
[13] O. Blanchard, P. Glorennec, F. Mercier, N. Bonvallot, C. Chevrier, O. Ramalho, C. Mandin, B. L. Bot Semivolatile, Organic compounds in indoor air and settled dust in 30 French dwellings, Environ. Sci. Technol. 48 (2014) 3959-3969.
[14] R. E. Dodson, L. J. Perovich, A. Covaci, N. Van den Eede, A. C. Ionas, A. C. Dirtu, J. G. Brody, R. A. Rudel, After the PBDE phase-out: a broad suite of flame retardants in repeat house dust samples from California, Environ. Sci. Technol. 46 (13) (2012) 056-13066.
[15] S. Huber, L. S. Haug, M. Schlabach, Per- and polyfluorinated compounds in house dust and indoor air from northern Norway—a pilot study, Chemosphere 84 (2011) 1686-1693.
[16] M. Shoeib, T. Harner, G. M. Webster, E. Sverko, Y. Cheng, Legacy and current-use flame retardants in house dust from Vancouver, Canada, Environ. Pollut. 169 (2012) 175-182.
[17] T. Geens, L. Roosens, H. Neels, A. Covaci, Assessment of human exposure to bisphenol-A, triclosan and tetrabromobisphenol-A through indoor dust intake in Belgium, Chemosphere 76 (2009) 755-760.
[18] N. H. Schebb, B. Inceoglu, K. C. C. Ahn Morisseau, S. J. B. D. Gee Hammock, Investigation of human exposure to triclocarban after showering and preliminary evaluation of its biological effects, Environ. Sci. Technol. 45 (2011) 3109-3115.
[19] P. D. Darbre, P. W. Harvey, Paraben esters: review of recent studies of endocrine toxicity, absorption, esterase and human exposure, and discussion of potential human health risks, J. Appl. Toxicol. 28 (2008) 561-578.
[20] J. Tang, K. Amin Usmani, E. Hodgson, R. L. Rose, In vitro metabolism of fipronil by human and rat cytochrome P450 and its interactions with testosterone and diazepam, Chem.-Biol. Interact. 147 (2004) 319-329.
[21] C. M. Butt, H. M. Stapleton, Inhibition of thyroid hormone sulfotransferase activity by brominated flame retardants and halogenated phenolics, Chem. Res. Toxicol. 26 (2013) 1692-1702.
[22] D. E. Buttke, K. Sircar, C. Martin, Exposures to endocrine-disrupting chemicals and age of menarche in adolescent girls in NHANES (2003-2008), Environ. Health Perspect. 120 (2012) 1613.
[23] P. Tarnow, T. Tralau, D. Hunecke, A. Luch, Effects of triclocarban on the transcription of estrogen, androgen and aryl hydrocarbon receptor responsive genes in human breast cancer cells, Toxicol. in Vitro 27 (2013) 1467-1475.
A. Salamova, Y. Ma, M. Venier, R. A. Hites, High levels of organophosphate flame retardants in the great lakes atmosphere, Environ. Sci. Technol. Lett. 1 (2013) 8-14.
[24] Build Cleaner Structures with Antimicrobial Flooring Product Technologies & More. Microban International. Retrieved from: <http://www.microban.com/what-we-do/by-product/building-materialshome-improvement>; 2014 [09.26.14].
[25] Biomonitoring California Designated Chemicals June 2014. California Environmental Contaminant Biomonitoring Program. Retrieved from biomonitoring.ca.gov, DesignatedChemicalsList June2014.pdf>; 2014 [09.22.14].
[26] Biomonitoring California Priority Chemicals June 2014. California Environmental Contaminant
[27] Biomonitoring Program. Retrieved from: biomonitoring.ca.gov, PriorityChemicals List June2014.pdf>; 2014 [09.22.14].
[28] Hartmann, E. M., D. R. Colquhoun, K. J. Schwab, and R. U. Halden. 2015. Absolute Quantification of Norovirus Capsid Protein in Food, Water, and Soil Using Synthetic Peptides with Electrospray and MALDI Mass Spectrometry. J. Hazardous Materials 286: 525-532.
[29] Rittmann, B. E., R. Krajmalnik-Brown, and R. U. Halden. 2008. Pre-genomic, Genomic and Post-genomic Study of Microbial Communities Involved in Bioenergy. Nature Microbiology Review 6(8):604-612. PMI D 18604223.

What is claimed is:

1. A method of sample collection for monitoring air quality in a structure comprising:
circulating indoor air through HVAC ductwork;
filtering the indoor air to remove a portion of air contaminants;
generating an atmospheric condensate from the filtered air;
collecting the atmospheric condensate in a collection vessel; and
conducting an analysis of the atmospheric condensate.

2. The method of claim 1 further comprising operating a cold trap to collect the condensate.

3. The method of claim 2 further comprising operating an air pump to pull air through the structure and out into the environment.

4. The method of claim 2 further comprising filling the cold trap with solvent.

5. The method of claim 2 further comprising incorporating a glass fiber filter to capture particulate contaminants into the cold trap.

6. The method of claim 1, wherein conducting an analysis of the atmospheric condensate comprises analyzing the atmospheric condensate for at least one member from a group of: gaseous chemicals, particulates, chemical aerosols, biological aerosols, aerosol-borne products, and microorganisms, wherein said microorganisms comprise whole microorganisms, constitutive parts and/or constituents of microorganisms, and combinations thereof.

7. The method of claim 6, wherein chemical aerosols comprise at least one of the following: chemical elements, chemical molecules, organic compounds, organometals, and inorganic compounds.

8. The method of claim 6, wherein biological aerosols comprise at least one of the following: microorganisms in whole or in part, including viruses, bacteria, yeasts, fungi, Archaea, prions, DNA, RNA, organelles, eukaryotic cells, natural and genetically modified cells.

9. The method of claim 6, wherein the aerosol-borne products comprise atmospheric moisture.

10. The method of claim 1 further comprising operating a heat exchange module to generate the atmospheric condensate.

11. The method of claim, 10 wherein operating a heat exchange module comprises using cooling coils of an HVAC system as the heat exchange module.

12. The method of claim 1, furthering comprising transporting air to be sampled through a condensate evacuation spout of an HVAC system, and using a cold trap as the collection vessel to collect the air to be sampled with the atmospheric condensate.

13. The method of claim 1, further comprises introducing a solvent into the collection vessel for capturing contaminants in the air to be sampled, wherein the solvent comprises at least one member from a group consisting of: condensed atmospheric moisture, polar solvents, nonpolar solvents, ionic liquids, water, and buffered solution.

14. The method of claim 13, wherein the solvent comprises at least one member of a group consisting of: methanol, ethanol, propanol, butanol, pentanol, hexanol, heptanol, octanol, pentane, hexane, heptane, octane, DSMO, trichloroethene, and dichloromethane.

15. The method of claim 1, further comprising using a condensate evacuation spout to collect the atmospheric condensate.

16. The method of claim 1, further comprising using an ultraclean glass jar as the collection vessel.

17. The method of claim 1, further comprising filtering the atmospheric condensate through a filter comprising at least one glass fiber membrane.

18. The method of claim 17, further comprising arranging the glass fiber membranes in decreasing pore size diameter.

19. The method of claim 18, wherein the first glass fiber membrane through which the atmospheric condensate passes comprises a larger pore size diameter than succeeding glass fiber membrane(s) through which the atmospheric condensate passes.

20. The method of claim 18, wherein the last glass fiber membrane that the atmospheric condensate passes through comprises pores of about 1.0 to about 0.8 μm in diameter.

21. The method of claim 18, wherein the last glass fiber membrane that the atmospheric condensate passes through comprises pores of about 0.6 to about 0.8 μm in diameter.

22. A method of sample collection for assessment of air quality in a building comprising:
circulating indoor air through HVAC ductwork;
filtering the indoor air to remove a portion of air contaminants;
generating an atmospheric condensate from the filtered air;
introducing solvent into a cold trap;
collecting the atmospheric condensate in the cold trap;
incorporating a glass fiber filter to capture particulate contaminants into the cold trap;
operating a calibrated air pump to pull air through the building and out into the environment; and
conducting an analysis of the atmospheric condensate.

23. A method of assessment of human health threats and human activities in a building comprising:
circulating indoor air through HVAC ductwork;
capturing airborne agents by generating an atmospheric condensate from the air;
introducing an optional preservative into the condensate;
collecting the atmospheric condensate in the cold trap;
conducting an analysis of agents in the atmospheric condensate; and
assessing human health threats and human activities in response to the analysis.

24. A method of monitoring air quality in a structure comprising:
circulating indoor air through HVAC ductwork;
filtering the indoor air to remove a portion of air contaminants;
generating an atmospheric condensate from the filtered air;
periodically collecting the atmospheric condensate in a collection vessel; and
periodically conducting an analysis of the atmospheric condensate to detect the presence of biological agents.

25. The method of claim 24 further including introducing a preservative into the condensate.
